(19) United States
(12) Patent Application Publication
(10) Pub. No.: US 2009/0220438 A1
(43) Pub. Date: Sep. 3, 2009

(54) CHROMEN-4-ONE DERIVATIVES AS SELF-TANNING SUBSTANCE

(75) Inventors: Christophe Carola, Heidelberg (DE); Anne Toullec, Darmstadt (DE); Herwig Buchholz, Frankfurt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GMBH, Darmstadt (DE)

(21) Appl. No.: 12/162,527

(22) PCT Filed: Jan. 9, 2007
(86) PCT No.: PCT/EP2007/000106

§ 371 Date: Apr. 20, 2009

(30) Foreign Application Priority Data
Jan. 31, 2006 (DE) 10 2006 004 327.8

(51) Int. Cl. A61K 8/49; C07D 311/04; A61Q 17/04

(52) U.S. Cl. 424/59; 549/401; 514/456

(57) ABSTRACT

The invention relates to the use of compounds of the formula (I), where R¹ and R² may be identical or different and stand for H, OH, OCH₃, CH₃, CH₂OH or CH₂OCH₃, R³ stands for H or straight-chain or branched C₁- to C₂₀-alkyl groups, R⁴ stands for H or OR⁸, R⁵ and R⁶ may be identical or different and are selected from —H, —OH, straight-chain or branched C₁ to C₂₀-alkyl groups, straight-chain or branched C₃- to C₂₀-alkenyl groups, straight-chain or branched C₁- to C₂₀-hydroxyalkyl groups, where the hydroxyl group may be bonded to a primary or secondary carbon atom of the chain, and furthermore the allyl chain may also be interrupted by oxygen, and R⁸ stands for H or straight-chain or branched C₁- to C₂₀-alkyl groups, with the proviso that R² does not denote H if R¹ denotes CH₃, as self-tanning agents, and to compounds and the preparation thereof.

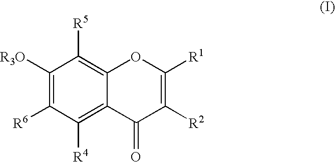

The present invention relates to the use of chromen-4-one derivatives as self-tanning active compounds and to novel chromen-4-one derivatives and the preparation thereof. The invention furthermore relates to compositions having an active content of chromen-4-one derivatives of this type.

The trend away from fashionable paleness to “healthy, sportily brown skin” has been uninterrupted for years. In order to achieve this, people expose their skin to sunlight since this causes pigmentation due to melanin formation. However, the UV radiation of sunlight also has a damaging effect on the skin. Besides acute damage (sunburn), long-term damage occurs, on excessive irradiation with light from the UVB region (wavelength 280-320 nm), such as, for example, an increased risk of developing skin cancer. In addition, excessive exposure to UVB and UVA radiation (wavelength: 320-400 nm) results in a weakening of the elastic and collagenic fibres of the connective tissue. This results in numerous phototoxic and photoallergic reactions and results in premature skin ageing.

Natural protection against the adverse consequences of sunlight is offered by tanning (pigmentation) of the skin. In its lowermost layer, the basal layer, the epidermis contains individual pigment-forming cells, the melanocytes, in addition to the basal cells. UV light stimulates the production of melanin in these cells, which is transported into the keratinocytes, where it becomes visible as a brown skin colour.

This pigment formation starting from the amino acid tyrosine is initiated predominantly by UVB radiation and is referred to as “indirect pigmentation”. Its development proceeds over a number of days; the resultant suntan lasts for a few weeks.

In the case of “direct pigmentation”, which commences with solar irradiation, predominantly colourless melanin precursors are oxidised by UVA radiation to dark-coloured melanin. Since this oxidation is reversible, it results in skin tanning which only lasts briefly.

Artificial tanning of the skin can be produced externally with the aid of make-up and orally by taking carotenoids.

Much more popular, however, is artificial tanning of the skin which can be achieved by application of so-called self-tanning agents. These compounds have, as chemical structural feature, keto or aldehyde groups in the vicinity of alcohol functions. These ketols or aldols belong predominantly to the sugars class of substances. A self-tanning substance which is employed particularly frequently is 1,3-dihydroxyacetone (DHA) or erythrulose (1,3,4-trihydroxy-2-butanone).

These compounds can be reacted with the proteins and amino acids of the horny layer of the skin in the sense of a Maillard reaction, where a reaction route which has not yet been fully clarified results in polymers which provide the skin with a brownish hue. This reaction is complete after about 4 to 6 hours. The tan achieved in this way cannot be washed off and is only removed with the normal skin desquamation. However, these coloured products do not themselves have UV-absorbent properties, meaning that additional sun protection (clothing, hat, UV filter) is necessary on exposure to the sun. In contrast to “sun-tanned” skin, skin tanned in this way is not protected against sunburn.

The object of the present invention is therefore to overcome disadvantages of the prior art and to find ways of preparing cosmetic and dermatological self-tanning formulations which enhance the natural tanning of the skin by increased melanin synthesis and at the same time result in improved inherent skin protection or sun protection, in particular against UVB radiation.

Surprisingly, it has now been found that certain chromen-4-one derivatives (chromone derivatives) are suitable as self-tanning active compounds. In addition, these chromone derivatives also have protection against UV radiation.

For the purposes of the invention, the term self-tanning active compound is used synonymously with self-tanning substance or self-tanner substance.

The same applications of structurally different compounds are known from the literature:

DE 10341663 describes cyclohexene derivatives for use on the skin or hair for enhancing skin tanning and melanin synthesis in the skin or hair.

Similar applications of structurally related compounds are known from the literature:

Compositions for topical use which comprise chromone derivatives, such as, for example, chromone, 7-hydroxychromone, 7-methoxychromone, 5,7-dihydroxy-2-methylchromone, 3-methyl-2-butenyloxychromone, 3-acetyl-5,7-dihydroxy-2-methylchromone, 5-hydroxychromone, n-pentyl 7-methoxychromone-2-carboxylate, n-undecyl 5-methoxychromone-2-carboxylate, 5-hydroxy-7-methoxy-2-methylchromone, 7-methoxychromone-2-carboxylic acid, n-pentylchromone-2-carboxylic acid, 5-methoxychromone and chromone-2-carboxylic acid, are known from Japanese Patent Application JP 05/301813. The chromone derivatives act as skin-tolerated tyrosinase inhibitors which reduce hyperpigmentation of the skin.

Japanese Patent Application JP 09/188,608 discloses the use of substituted chromone derivatives, such as, in particular, 5,7-dihydroxychromones, 7-methoxychromones, 5-hydroxy-7-methoxy-2-methylchromone and 5-hydroxy-2-methylchromone, as active compound against grey hair. The action here is attributed to activation of the cells which form coloured pigments and the increase in melanogenesis.

The present invention therefore relates firstly to the use of compounds of the formula I

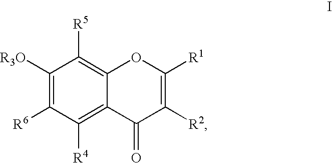

where
R¹ and R² may be identical or different and stand for H, OH, OCH₃, CH₃, CH₂OH or CH₂OCH₃,
R³ stands for H or straight-chain or branched C₁- to C₂₀-alkyl groups,
R⁴ stands for H or OR⁸,
R⁵ and R⁶ may be identical or different and are selected from
   —H, —OH,
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain, and furthermore the alkyl chain may also be interrupted by oxygen, and
R⁸ stands for H or straight-chain or branched C₁- to C₂₀-alkyl groups, with the proviso that R² does not denote H if R¹ denotes methyl, as self-tanning substance.

For the purposes of the present invention, the term “compound of the formula I” basically also encompasses the salts of the particular compounds of the formula I. The preferred salts here include, in particular, alkali and alkaline-earth metal salts and ammonium salts, but in particular sodium and potassium salts.

It is preferred for the use according to the invention for the compounds to be characterised in that R¹ stands for CH₂OCH₃, CH₂OH or CH₃.

With respect to the desired property profile, it has furthermore proven preferred for the compounds to be characterised in that R² stands for H, OH or OCH₃. R² particularly preferably stands for OH or OCH₃. Very particularly preferred self-tanner substances are compounds of the formula I in which R² stands for OCH₃.

It is additionally preferred for the compounds used in accordance with the invention to be characterised in that R³ stands for H and R⁴ stands for H or OH.

It is additionally preferred for the compounds used in accordance with the invention to be characterised in that R⁵ denotes OH or H and R⁶ denotes H.

Especial preference is given in accordance with the invention to the use as self-tanning substance of compounds of the formula I selected from the compounds having the formulae Ia-Ig:

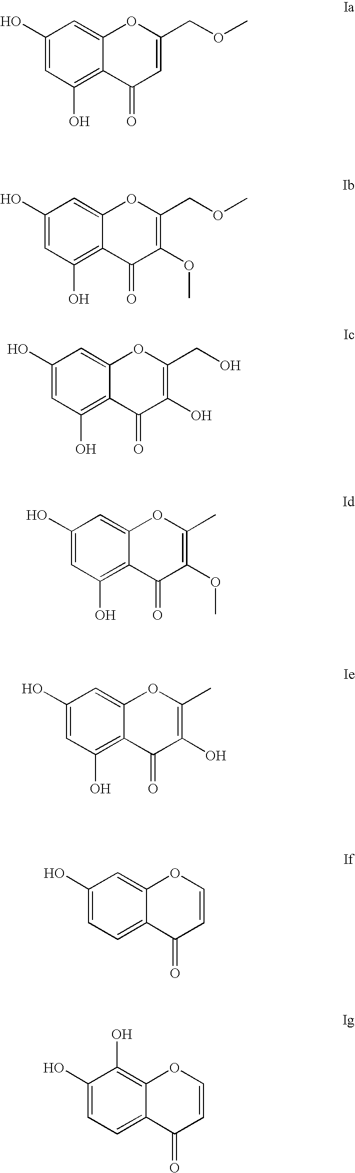

Very particularly preferred self-tanning active compounds are compounds Id and If.

The invention furthermore relates to the compounds of the formula I

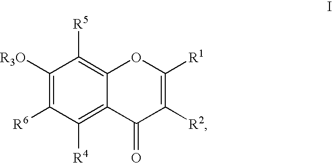

where
R¹ denotes CH₂OH or CH₂OCH₃,
R² denotes OH or OCH₃,
R³ stands for H or straight-chain or branched C₁- to C₂₀-alkyl groups,
R⁴ stands for H or OR⁸,
R⁵ and R⁶ may be identical or different and are selected from
   —H, —OH,
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain, and furthermore the alkyl chain may also be interrupted by oxygen, and
      R⁸ stands for H or straight-chain or branched C₁- to C₂₀-alkyl groups. Straight-chain or branched alkyl groups having 1 to 20 C atoms, preferably having 1, 2, 3, 4, 5 or 6 C atoms, are, for example, methyl, ethyl, isopropyl, n-propyl, isobutyl, n-butyl, tert-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosanyl, and isomers thereof.

Alkenyl groups are groups of the empirical formula CₙH₂ₙ₋₁, where n=3 to 20.

In a variant of the invention, it is preferred for R³ in formula I to stand for H.

In a variant of the invention, it is preferred for R⁴ to stand for H or OH.

In a variant of the invention, it is preferred for R⁵ to stand for H or OH and R⁶ to stand for H.

Preferred novel compounds of the formula I are the compounds of the formulae Ib and Ic:

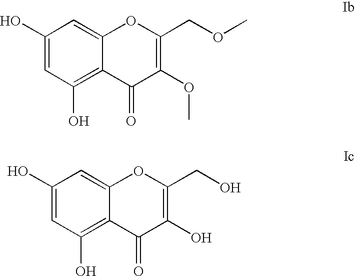

As a selection from the general formula I, defined above for the use, the novel compounds of the formula I are preferably to be used as cosmetic active compounds, particularly preferably as self-tanning substances.

The invention therefore furthermore relates to a composition comprising at least one compound of the formula I

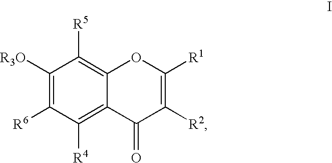

where
R¹ denotes CH₂OH or CH₂OCH₃,
R² denotes OH or OCH₃,
R³ stands for H or straight-chain or branched C₁- to C₂₀-alkyl groups,
R⁴ stands for H or OR⁸,
R⁵ and R⁶ may be identical or different and are selected from
   —H, —OH,
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain, and furthermore the alkyl chain may also be interrupted by oxygen, and
      R⁸ stands for H or straight-chain or branched C₁- to C₂₀-alkyl groups, and at least one vehicle which is suitable for topical applications.

The present invention furthermore relates to a self-tanning composition comprising at least one compound of the formula I

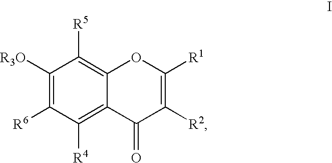

where
R¹ and R² may be identical or different and stand for H, OH, OCH₃, CH₃, CH₂OH or CH₂OCH₃,
R³ stands for H or straight-chain or branched C₁- to C₂₀-alkyl groups,
R⁴ stands for H or OR⁸,
R⁵ and R⁶ may be identical or different and are selected from
   —H, —OH,
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain, and furthermore the alkyl chain may also be interrupted by oxygen, and
   R⁸ stands for H or straight-chain or branched C₁- to C₂₀-alkyl groups,
      with the proviso that R² does not denote H if R¹ denotes methyl, and at least one vehicle which is suitable for topical applications.

Uses which are preferred in accordance with the invention of the compounds of the formula I containing radicals as described above or of compositions comprising at least one compound of the formula I, as described above, are, in particular, the use as self-tanning substance and/or for increasing melanin synthesis in the skin and/or for protecting the skin against harmful UV rays.

Uses which are preferred in accordance with the invention of the compounds of the formulae Ia to Ig or of compositions comprising at least one compound of the formulae Ia to Ig are likewise the use for increasing melanin synthesis in the skin.

The present invention also relates here in each case to the use of the compounds of the formula I, as defined above, for the preparation of compositions for the uses indicated above.

The content of the compounds of the formula I in the composition is between 0.01 and 10% by weight, advantageously between 0.1 and 5% by weight, based on the total weight of the composition. The person skilled in the art will have absolutely no difficulties in selecting the amounts correspondingly depending on the intended action of the composition.

For the purposes of the invention, the term composition is used synonymously with the term formulation.

The compositions here are usually either compositions which can be used topically, for example cosmetic or dermatological formulations, or foods or food supplements. In this case, the compositions comprise a cosmetically or dermatologically or food-suitable vehicle and, depending on the desired property profile, optionally further suitable ingredients.

The use according to the invention of chromen-4-one derivatives of the formula I in compositions offers, inter alia, protection against damage caused by UV radiation.

The compounds of the formula I, as described above, can also occur dispersed in finely divided form in the compositions according to the invention. This produces a slow-release effect, i.e. controlled or delayed penetration into the skin or controlled distribution of the chromen-4-one derivative on or in the skin.

The compositions according to the invention may include or comprise, essentially consist of or consist of the said necessary or optional constituents. All compounds or components which can be used in the compositions are either known and commercially available or can be synthesised by known processes.

The cosmetic or dermatological composition or self-tanner composition, as described above, can preferably comprise at least one further self-tanner substance. This may further enhance or modify the tanning effect. Self-tanning substances which can be employed, inter alia, are:

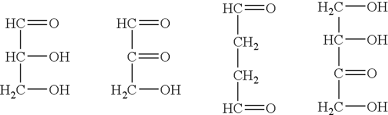

glycerolaldehyde hydroxymethylglyoxal γ-dialdehyde erythrulose (glyceraldehyde)

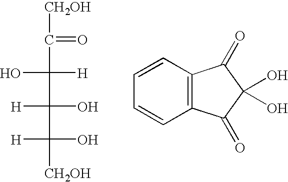

6-aldo-D-fructose ninhydrin,
5-hydroxy-1,4-naphthoquinone (juglone), which is extracted from the shells of fresh walnuts,

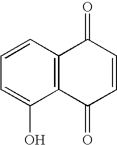

5-hydroxy-1,4-naphthoquinone (juglone),
2-hydroxy-1,4-naphthoquinone (lawsone), which occurs in henna leaves,

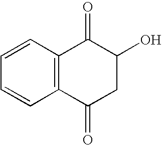

2-hydroxy-1,4-naphthoquinone (lawsone),

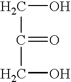

1,3-dihydroxyacetone (DHA),
dihydroxyacetone phosphate, glyceraldehyde phosphate and erythrose.

The self-tanner substances employed are preferably 1,3-dihydroxyacetone (DHA), glyceraldehyde, dihydroxyacetone phosphate, glyceraldehyde phosphate, erythrose or 1,3,4-trihydroxy-2-butanone (erythrulose).

Erythrulose or 1,3-dihydroxyacetone (DHA), a trifunctional ketosugar which occurs in the human body, and derivatives thereof may particularly preferably be present. DHA may very particularly preferably be present.

The composition according to the invention, which combines a self-tanning substance and a chromone of the formula I, has the following advantages over a self-tanning product without addition of chromone:
stabilisation of the self-tanning substance to oxygen (on the skin and in the product),
acceleration of the tanning reaction,
extension of the tanning reaction owing to the indirect tanning reaction (UV-free tanning extension),
enhancement of the tanning reaction,
the tanning achieved is similar to natural tanning.

In a further preferred embodiment of the present invention, the composition comprises one or more antioxidants and/or one or more vitamins.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the general formula

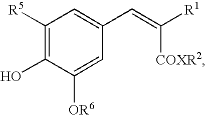

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes linear or branched alkyl having 1 to 8 C atoms or linear or branched alkoxy having 1 to 8 C atoms, and
R⁶ denotes linear or branched alkyl having 1 to 8 C atoms, preferably derivatives of 2-(4-hydroxy-3,5-dimethoxybenzylidene)malonic acid, particularly preferably bis(2-ethylhexyl) 2-(4-hydroxy-3,5-dimethoxybenzylidene)malonate (for example Oxynex® ST Liquid).

Mixtures of antioxidants are likewise suitable for use in the cosmetic compositions according to the invention. Known and commercial mixtures are, for example, mixtures comprising, as active compounds, lecithin, L-(+)ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Anti-oxidants of this type are usually employed with compounds of the formula I in such formulations in ratios in the range from 1000:1 to 1:1000, preferably in amounts of 100:1 to 1:100.

The compositions according to the invention may comprise vitamins as further ingredients, as described above. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin C and derivatives thereof, DL-α-tocopherol, tocopherol E acetate, nicotinic acid, pantothenic acid and biotin. Vitamins are usually employed here with compounds of the formulae Ia-m in ratios in the range from 1000:1 to 1:1000, preferably in amounts of 100:1 to 1:100.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Pagan-ga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology & Medicine 2001, 31(7), 869-881, are investigating the pH dependence of the antioxidant action of hydroxyflavones. Quercetin exhibits the greatest activity amongst the structures investigated over the entire pH range.

Particular advantages of the compositions according to the invention here, besides the tanning action, are also the antioxidant action and the good tolerance by the skin. In addition, preferred compounds of those described here are colourless or only weakly coloured and thus only result in slight discolourations of the compositions, or none at all. Of particular advantage is the particular action profile of the compounds of the formula I, which is evident in the DPPH assay from a high capacity for scavenging free radicals (EC₅₀), a delayed action (TEC₅₀>120 min) and thus a moderate to high anti-free-radical efficiency (AE). In addition, the compounds of the formula I combine antioxidative properties with UV absorption in the UV-B region in the molecule.

In order that the compounds of the formula I are able to develop their positive action on the skin particularly well, it may be preferred to allow the compounds of the formula I, as described above, to penetrate into deeper skin layers. Several possibilities are available for this purpose. Firstly, the compounds of the formula I can have an adequate lipophilicity in order to be able to penetrate through the outer skin layer into epidermal layers. As a further possibility, corresponding transport agents, for example liposomes, which enable transport of the compounds of the formula I through the outer skin layers may also be provided in the composition. Finally, systemic transport of the compounds of the formula I is also conceivable. The composition is then designed, for example, in such a way that it is suitable for oral administration.

Owing to these properties, the compositions according to the invention are, in general, suitable for immune protection and for the protection of DNA and RNA. In particular, the compositions are suitable for the protection of DNA and RNA against oxidative attack, against free radicals and against damage due to radiation, in particular UV radiation. A further advantage of the compositions according to the invention is cell protection, in particular protection of Langerhans cells against damage due to the above-mentioned influences. All these uses and the use of the compounds of the formula I for the preparation of compositions which can be employed correspondingly are expressly also a subject-matter of the present invention.

In a further variant of the invention, it may be advantageous for the compositions described also to comprise UV filters in addition to the compounds of the formula I.

In principle, all UV filters are suitable for combination with the compounds of the formula I. Particular preference is given to UV filters whose physiological acceptability has already been demonstrated. Both for UVA and UVB filters, there are many proven substances known from the specialist literature, for example

benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for exampie Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25),
phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid,
and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul®UVA Plus, BASF).

The compounds mentioned in the list should only be regarded as examples. It is of course also possible to use other UV filters.

These organic UV filters are generally incorporated into cosmetic formulations in an amount of 0.5 to 10 percent by weight, preferably 1-8% by weight.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and approximately 6% of methyl[2-[p-[2,2-bis(ethoxycarbonyl]vinyl]phenoxy]-1-methyleneethyl] and approximately 1.5% of methyl[3-[p-[2,2-bis(ethoxycarbonyl)vinyl)phenoxy)propenyl) and 0.1 to 0.4% of (methylhydrogen]silylene]] (n≈60) (CAS No. 207 574-74-1)
2,2′-methylenebis(6-(2H-benzotriazol-2-yl)-4-(1,1,3,3-tetramethylbutyl)phenol) (CAS No. 103 597-45-1)
2,2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxyl]phenyl}-6-(4-methoxyphenyl)-1,3,5-triazine (CAS No. 103 597-45-, 187 393-00-6).

Further suitable UV filters are also methoxyflavones corresponding to the earlier German patent application DE 10232595.2.

Organic UV filters are generally incorporated into cosmetic formulations in an amount of 0.5 to 20 percent by weight, preferably 1-15% by weight.

Conceivable inorganic UV filters are those from the group of the titanium dioxides, such as, for example, coated titanium dioxide (for example Eusolex® T-2000, Eusolex® T-AQUA), zinc oxides (for example Sachtotec®), iron oxides or also cerium oxides. These inorganic UV filters are generally incorporated into cosmetic compositions in an amount of 0.5 to 20 percent by weight, preferably 2-10%.

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and potassium, sodium and triethanolamine salts thereof.

The protective action against damaging effects of UV radiation can be optimised by combining one or more compounds of the formula I with further UV filters.

Optimised compositions may comprise, for example, the combination of the organic UV filter 4′-methoxy-6-hydroxyflavone with 1-(4-tert-butylphenyl)-3-(4-methoxyphenyl)propane-1,3-dione and 3-(4′-methylbenzylidene)-dl-camphor. This combination gives rise to broad-band protection, which can be supplemented by the addition of inorganic UV filters, such as titanium dioxide microparticles.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin is repeatedly being discussed in the literature. The encapsulation of the corresponding substances which is proposed here suppresses this effect.
In general, encapsulation of individual UV filters or other ingredients enables composition problems caused by the interaction of individual composition constituents with one another, such as crystallisation processes, precipitation and agglomerate formation, to be avoided since the interaction is suppressed.

It is therefore preferred in accordance with the invention for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active compound (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules in compositions according to the invention are preferably present in amounts which ensure that the encapsulated UV filters are present in the composition in the above-indicated amounts.

The compositions, as described above, may also comprise further skin-protecting or skin-care active compounds. These may in principle be any active compounds known to the person skilled in the art.

In an embodiment of the present invention, particularly preferred active compounds are pyrimidinecarboxylic acids and/or aryl oximes.

Pyrimidinecarboxylic acids occur in halophilic microorganisms and play a role in the osmoregulation of these organisms (E. A. Galinski et al., Eur. J. Biochem., 149 (1985) pages 135-139). Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacterial cells. Pharmaceutical products, such as non-glycosylated, pharmaceutically active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-up, care creams and sunscreen preparations.

Of the aryl oximes, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for therapy of psoriasis, various forms of eczema, irritative and toxic dermatitis, UV dermatitis and further allergic and/or inflammatory diseases of the skin and integumentary appendages. Compositions according to the invention which, in addition to the compound of the formulae Ia to Ig, additionally comprise an aryl oxime, preferably 2-hydroxy-5-methyllaurophenone oxime, exhibit surprising antiinflammatory suitability. The compositions here preferably comprise 0.01 to 10% by weight of the aryl oxime, it being particularly preferred for the composition to comprise 0.05 to 5% by weight of aryl oxime.

All compounds or components which can be used in the compositions are either known and commercially available or can be synthesised by known processes. The preparation of the novel compounds of the formula I, as described above, is described below.

The at least one compound of the formula I can be incorporated into cosmetic or dermatological compositions in the usual manner. Suitable compositions are those for external use, for example in the form of a cream, lotion or gel or in the form of a solution which can be sprayed onto the skin. Suitable for internal use are administration forms such as capsules, dragees, powders, tablet solutions or solutions.

Use forms of the compositions according to the invention that may be mentioned are, for example: solutions, suspensions, emulsions, PIT emulsions, pastes, ointments, gels, creams, lotions, powders, soaps, surfactant-containing cleansing preparations, oils, aerosols and sprays. Examples of other use forms are sticks, shampoos and shower compositions. Any desired customary vehicles, assistants and, if desired, further active compounds may be added to the composition.

Preferred assistants originate from the group of the preservatives, stabilisers, solubilisers, colorants and odour improvers.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, corn oil, olive oil, castor oil and sesame oil, glycerol fatty acid esters, polyethylene glycols and fatty acid esters of sorbitan, or mixtures of these substances.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic use forms are also lipsticks, lip-care sticks, mascara, eyeliner, eyeshadow, rouge, powder make-up, emulsion make-up and wax make-up, and sunscreen, pre-sun and after-sun preparations.

The preferred composition forms according to the invention include, in particular, emulsions.

Emulsions according to the invention are advantageous and comprise, for example, the said fats, oils, waxes and other fatty substances, as well as water and an emulsifier, as usually used for a composition of this type. The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase can furthermore advantageously be selected from the group of the branched and unbranched hydrocarbons and hydrocarbon waxes, silicone oils, dialkyl ethers, or the group of the saturated or unsaturated, branched or unbranched alcohols, and fatty acid triglycerides, specifically triglycerol esters of saturated and/or unsaturated, branched and/or unbranched alkanecarboxylic acids having a chain length of 8 to 24, in particular 12-18, C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane, poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol or glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984 or 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

In a preferred embodiment, the compositions according to the invention comprise hydrophilic surfactants.

The hydrophilic surfactants are preferably selected from the group of the alkylglucosides, acyl lactylates, betaines and coconut amphoacetates.

Alkylglycosides which are particularly advantageously used for the purposes of the invention are selected from the group octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and assistants or mixtures which are distinguished by an effective content of the active compounds used in accordance with the invention, for example Plantaren® 1200 (Henkel KGaA), Oramix® NS 10 (Seppic).

The acyllactylates are themselves advantageously selected from the group of the substances which are distinguished by the structural formula

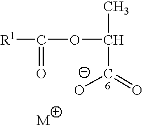

where R¹ denotes a branched or unbranched alkyl radical having 1 to 30 carbon atoms, and M⁺ is selected from the group of the alkali metal ions and the group of the ammonium ions which are substituted by one or more alkyl and/or one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group of the substances which are distinguished by the structural formula

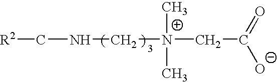

where R² denotes a branched or unbranched alkyl radical having 1 to 30 carbon atoms.

R² particularly advantageously denotes a branched or unbranched alkyl radical having 6 to 12 carbon atoms.

For example, capramidopropylbetaine, for example the product Tego® Betain 810 from Th. Goldschmidt AG, is advantageous.

A coconut amphoacetate which is advantageous for the purposes of the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological compositions according to the invention are applied in sufficient amount to the skin and/or hair, preferably to the skin, in the usual manner for cosmetics.

Cosmetic and dermatological compositions according to the invention may exist in various forms. Thus, they may be, for example, a solution, a waterfree composition, an emulsion or microemulsion of the water-in-oil (W/O) or oil-in-water (O/W) type, a multiple emulsion, for example of the water-inoil-in-water (W/O/W) type, a gel, a solid stick, an ointment or an aerosol. It is also advantageous to administer ectoines in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

Co-emulsifiers which are advantageous according to the invention are, for example, O/W emulsifiers, principally from the group of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or in the case of isoalkyl derivatives, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Optional W/O emulsifiers, but ones which may nevertheless be advantageous for the purposes of the invention can be the following:

fatty alcohols having 8 to 30 carbon atoms, monoglycerol esters of saturated and/or unsaturated, branched and/or unbranched alkanecarboxylic acids having a chain length of 8 to 24, in particular 12-18, C atoms, diglycerol esters of saturated and/or unsaturated, branched and/or unbranched alkanecarboxylic acids having a chain length of 8 to 24, in particular 12-18, C atoms, monoglycerol ethers of saturated and/or unsaturated, branched and/or unbranched alcohols having a chain length of 8 to 24, in particular 12-18, C atoms, diglycerol ethers of saturated and/or unsaturated, branched and/or unbranched alcohols having a chain length of 8 to 24, in particular 12-18, C atoms, propylene glycol esters of saturated and/or unsaturated, branched and/or unbranched alkanecarboxylic acids having a chain length of 8 to 24, in particular 12-18, C atoms, and sorbitan esters of saturated and/or unsaturated, branched and/or unbranched alkanecarboxylic acids having a chain length of 8 to 24, in particular 12-18, C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate and glyceryl monocaprylate.

The composition according to the invention may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a composition is formulated as an aerosol, the customary propellants, such as alkanes, fluoroalkanes and chlorofluoroalkanes, are generally used.

The present invention furthermore relates to a process for the preparation of a composition which is characterised in that at least one compound of the formula I containing radicals as described above is preferably mixed with a vehicle which is suitable cosmetically or dermatologically or with a vehicle which is suitable for foods, and to the use of a compound of the formula I for the preparation of a composition.

The compositions according to the invention can be prepared here with the aid of techniques which are well known to the person skilled in the art.

The mixing can result in dissolution, emulsification or dispersal of the at least one compound of the formula I in the vehicle.

The compounds of the formula I, as defined above for the use according to the invention, can be obtained by cyclisation of a correspondingly substituted o-hydroxyacetophenone with an anhydride or with an acyl chloride under basic conditions. The acyl groups, i.e. on the one hand O-acyl groups formed by reaction of the free hydroxyl group with the anhydride or acyl chloride, on the other hand C-acyl groups in position 3 of the chromone system, can subsequently be removed. The reaction here can be carried out analogously to Kelly, T; Kim M. H.; J. Org. Chem. 1992, 57, 1593-97.

Alternatively, the free hydroxyl groups are acylated. A Baker-Venkatamaran rearrangement is subsequently carried out in basic conditions with subsequent ring closure under acidic conditions. Corresponding reactions, the adaptation of which to the compounds desired here presents the person skilled in the art with absolutely no difficulties, are known from patent application WO 2002/060889.

Conventional reactions on the ring system or derivatisation of the functional groups enables further derivatives of the formula I to be obtained. The reaction condition necessary for this purpose for such reactions, such as, for example, oxidations, reductions, transesterifications, etherifications, will easily be found by a person skilled in the art for syntheses of this type in the generally available literature on organic reactions.

The invention furthermore also relates to a process for the preparation of the compounds of the formula I

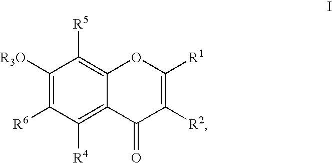

where
R¹ denotes CH₂OH or CH₂OCH₃,
R² denotes OH or OCH₃,
R³ stands for H or straight-chain or branched C₁- to C₂₀-alkyl groups,
R⁴ stands for H or OR⁸,
R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain, and furthermore the alkyl chain may also be interrupted by oxygen, and
   R⁸ stands for H or straight-chain or branched C₁- to C₂₀-alkyl groups, characterised in that
   an o-hydroxyacetophenone of the formula II

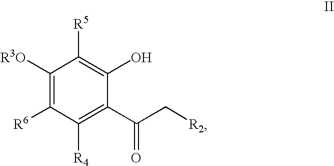

where the substituents R² to R⁶ have one of the meanings or preferred meanings described above,
is cyclised with methoxyacetyl chloride under basic conditions and the methyl ethers present are optionally cleaved.

The cyclisation is preferably carried out in a solvent selected from the group tetrahydrofuran, diethyl ether, diisopropyl ether, methyl tert-butyl ether and 1,4-dioxane.

The cyclisation is preferably carried out under inert-gas conditions. K₂CO₃ or another reagent from the group KO-tBu (potassium tert-butoxide), LiOH, KOH or DBU is advantageously introduced in pyridine at temperatures between −10° C. and 35° C., preferably at room temperature. The actual reaction temperature is between −10 and 175° C., preferably between 35 and 100° C. The reaction is particularly preferably carried out at 50° C. or 66° C.

The compounds of the formula I in which R¹ denotes CH₂—OH and R² denotes OH are preferably prepared by subsequent ether cleavage of the compounds of the formula I in which R¹ denotes CH₂—OCH₃ and R² denotes OCH₃.

It has also been found that compounds of the formula I, as described above, can have a stabilising action on the composition. On use in corresponding products, these therefore also remain stable for longer and do not change their appearance. In particular, the efficacy of the ingredients, for example vitamins, is retained even on extended use or extended storage. This is particularly advantageous, inter alia, in the case of compositions for protecting the skin against the action of UV rays, since these cosmetics are subjected to particularly high stresses by the UV radiation.

The positive actions of compounds of the formula I give rise to their particular suitability for use in cosmetic or pharmaceutical compositions, in particular in cosmetic compositions.

It has furthermore been found that the intermediates or precursors (such as, for example, 5,7-dihydroxy-3-(2-methoxyacetyl)-2-methoxymethyl-1-benzopyran-4-one (compound IIa)) of the compounds of the formula I according to the invention likewise have a tanning activity and can thus also be employed as tanning agents.

The properties of compounds of the formula I should likewise be regarded as positive for use in foods or as food supplements or as functional foods. The further explanations given for foods also apply correspondingly to food supplements and functional foods.

The foods which can be enriched with one or more compounds of the formula I in accordance with the present invention include all materials which are suitable for consumption by animals or consumption by humans, for example vitamins and provitamins thereof, fats, minerals or amino acids. (The foods may be solid, but also liquid, i.e. in the form of a beverage). The present invention accordingly furthermore relates to the use of a compound of the formula I as food additive for human or animal nutrition, and to compositions which are foods or food supplements and comprise corresponding vehicles.

Foods which can be enriched with one or more compounds of the formula I in accordance with the present invention are, for example, also foods which originate from a single natural source, such as, for example, sugar, unsweetened juice, squash or puree of a single plant species, such as, for example, unsweetened apple juice (for example also a mixture of different types of apple juice), grapefruit juice, orange juice, apple compote, apricot squash, tomato juice, tomato sauce, tomato puree, etc. Further examples of foods which can be enriched with one or more compounds of the formulae Ia to Ig in accordance with the present invention are corn or cereals from a single plant species and materials produced from plant species of this type, such as, for example, cereal syrup, rye flour, wheat flour or oat bran. Mixtures of foods of this type are also suitable for being enriched with one or more compounds of the formula I in accordance with the present invention, for example multivitamin preparations, mineral mixtures or sweetened juice. As further examples of foods which can be enriched with one or more compounds of the formula I in accordance with the present invention, mention may be made of food compositions, for example prepared cereals, biscuits, mixed drinks, foods prepared especially for children, such as yoghurt, diet foods, low-calorie foods or animal feeds.

The foods which can be enriched with one or more compounds of the formula I in accordance with the present invention thus include all edible combinations of carbohydrates, lipids, proteins, inorganic elements, trace elements, vitamins, water or active metabolites of plants and animals. The foods which can be enriched with one or more compounds of the formula I in accordance with the present invention are preferably administered orally, for example in the form of meals, pills, tablets, capsules, powders, syrup, solutions or suspensions.

The foods according to the invention enriched with one or more compounds of the formula I can be prepared with the aid of techniques which are well known to the person skilled in the art.

Due to their action, compounds of the formula I are also suitable as medicament ingredient.

The invention is explained in greater detail below with reference to examples. The invention can be carried out throughout the range claimed and is not restricted to the examples given here.

### EXAMPLES

### Example 1

### Preparation of 5,7-dihydroxy-2-methoxymethylchromen-4-one (Ia)

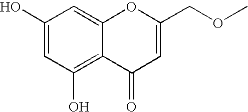

### Preparation of the precursor 5,7-dihydroxy-3-(2-methoxyacetyl)-2-methoxymethyl-1-benzopyran-4-one (IIa)

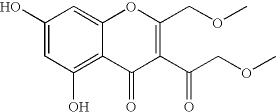

2,4,6-Trihydroxyacetophenone (10 g, 53.7 mmol) is dissolved in 100 ml of THF (tetrahydrofuran) at room temperature (RT) under an inert gas and with stirring. K₂CO₃ (30 g, 217.1 mmol) is added, and the yellow suspension is heated to 50° C. and stirred for 30 min. Methoxyacetyl chloride (10.3 ml, 112.8 mmol) is added dropwise to the warm suspension, and the mixture is heated under reflux, i.e. at 66° C., for a further 6 h. The solution is cooled and poured into ice-water. HCl (32%) is added for acidification, and the solution is then extracted with ethyl acetate. The organic phase is dried over Na₂SO₄, filtered and evaporated to give an oil, which then crystallises. The crystals are subsequently washed with dichloromethane.

Yield: 7.5 g (46%)

EI-MS: m/e: 294 M⁺

¹H NMR (250 MHz, DMSO): δ (ppm)=3.32 (s, 3H, CH₃ of 2-methoxymethyl); 3.34 (s, 3H, CH₃ of 3-methoxyacetyl); 4.39 (s, 2H, CH₂ of 2-methoxymethyl); 4.46 (s, 2H, CH₂ of 3-methoxyacetyl); 6.25 (d, 1H, J=2.0 Hz, H-8); 6.39 (d, 1H, J=2.0 Hz, H-6); 11.05 (s, 1H, OH-7); 12.21 (s, 1H, OH-5).

¹³C NMR (75 MHz, DMSO, protons decoupled): δ (ppm)=58.5 (CH₃); 58.6 (CH₃); 68.9 (CH₂); 77.7 (CH₂); 94.1 (CH); 99.5 (CH); 103.2 (Cquat.); 119.5 (Cquat.); 156.7 (Cquat.); 161.4 (Cquat.); 165.0 (Cquat.); 165.3 (Cquat.); 179.2 (Cquat.); 198.8 (Cquat.).

HR-MS: (C₁₄H₁₄O₇) calc.: 294.0739; found: 294.0736±1.1 ppm.

### Preparation of 5,7-dihydroxy-2-methoxymethyl-1-benzopyran-4-one (Ia)

5,7-Dihydroxy-3-(2-methoxyacetyl)-2-methoxymethylchromone (IIa) (1 g, 3.4 mmol) is suspended in 200 ml of water. K₂CO₃ (1.41 g, 10.2 mmol) is added at RT with stirring. The suspension is boiled under reflux for 1 h, and the yellow solution is then re-cooled to RT. Ice is added, and the solution is adjusted to pH=4 using 32% HCl. A white precipitate forms, which is filtered off and dried in vacuo at 40° C. The solid is recrystallised from dichloromethane in order to obtain a white product.

Yield: 550 mg (71%)

MP: 180° C.

EI-MS: m/e: 222 M⁺

¹H NMR (250 MHz, DMSO): δ (ppm)=3.39 (s, 3H, CH₃); 4.37 (s, 2H, CH₂); 6.22 (d, 1H, J=1.75 Hz, H-8); 6.26 (s, 1H, H-3); 6.37 (d, 1H, J=1.75 Hz, H-6); 10.85 (s, 1H, OH-7), 12.65 (s, 1H, OH-5).

¹³C NMR (75 MHz, DMSO, protons decoupled): δ (ppm)=58.3 (CH₃); 69.6 (CH₂); 93.8 (CH); 98.9 (CH); 103.9 (Cquat.); 107.3 (CH); 157.5 (Cquat.); 161.5 (Cquat.); 164.3 (Cquat.); 166.0 (Cquat.); 181.5 (Cquat.).

HR-MS: (C₁₁H₁₀O₅) calc.: 222.0528; found: 222.0524±1.9 ppm.

### Example 2

### Preparation of 5,7-dihydroxy-3-methoxy-2-methoxymethylchromen-4-one (Ib)

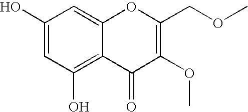

### Preparation of the precursor 2-methoxy-1-(2,4,6-trihydroxyphenyl)ethanone (IIb)

### (Friedel and Crafts)

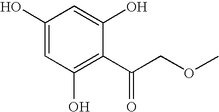

Phloroglucinol (1.3 g, 10 mmol) is dissolved in dry ether (50 ml) under an inert gas. Methoxyacetyl chloride (1.03 ml, 11.0 mmol) is added at RT with stirring, and the mixture is then cooled to 0° C. before AlCl₃ (5.47 g, 41 mmol) is added. The mixture is stirred overnight. The reaction mixture is poured into ice, and 32% HCl is then added in order to adjust the pH to 5-6. The solution is extracted with diethyl ether, and the combined extracts are washed with water and dried using Na₂SO₄, and the solvent is evaporated off under reduced pressure in order to give a white product, which is recrystallised from methanol.

Yield: 1.25 g (63%)

EI-MS: m/e: 198 M⁺

¹H NMR (250 MHz, DMSO): δ (ppm)=3.33 (s, 3H, CH₃); 4.58 (s, 2H, CH₂); 5.82 (s, 1H, H-3 and H-5); 10.40 (bs, 1H, OH-4); 12.13 (s, 2H, OH-2 and OH-6).

¹³C NMR (75 MHz, DMSO, protons decoupled): δ (ppm)=58.4 (CH₃), 77.0 (CH₂), 94.5 (CH), 102.4 (Cquat.), 163.9 (Cquat.), 164.8 (Cquat.), 201.2 (Cquat.).

HR-MS: (C₉H₁₀O₅) calc.: 198.0528; found: 198.0530±1.3 ppm.

### 5,7-Dihydroxy-3-methoxy-2-methoxymethyl-1-benzopyran-4-one (Ib)

2-Methoxy-1-(2,4,6-trihydroxyphenyl)ethanone (IIb) (10 g, 50.46 mmol) is dissolved in THF (250 ml) at RT with stirring and under an inert gas. K₂CO₃ (34.87 g, 252.3 mmol) is added, and the yellow suspension is stirred and heated at 50° C. for 1 h. Methoxyacetyl chloride (9.70 ml, 106 mmol) is added dropwise. The mixture is stirred for a further 4 h. The solution is subsequently cooled and poured into ice-water. HCl (32%) is added for neutralisation (pH 6), and the mixture is then extracted with ethyl acetate. The organic phase is dried using Na₂SO₄, filtered and evaporated to give a white-orange product. Recrystallisation is carried out from dichloromethane.

Yield: 4.2 g (33%)

MP: 173-174° C.

EI-MS: m/e: 252 M⁺

¹H NMR (250 MHz, DMSO): δ (ppm)=3.36 (s, 3H, CH₃ of 2-methoxymethyl); 3.81 (s, 3H, CH₃ of 3-methoxy); 4.45 (s, 2H, CH₂); 6.21 (d, 1H, J=2.0 Hz, H-8); 6.36 (d, 1H, J=2.0 Hz, H-6); 10.88 (s, 1H, OH-7); 12.44 (s, 1H, OH-5).

¹³C NMR (75 MHz, DMSO, protons decoupled): δ (ppm)=58.1 (CH₃); 60.8 (CH₃); 65.6 (CH₂); 93.7 (CH); 98.7 (CH); 104.6 (Cquat.); 139.6 (Cquat.); 152.7 (Cquat.); 161.4 (Cquat.); 164.3 (Cquat.); 177.5 (Cquat.).

HRMS: (C₁₂H₁₂O₆) calc.: 252.0633; found: 252.0636±1.2 ppm.

### Example 3

### Preparation of 2-hydroxymethyl-3,5,7-trihydroxymethylchromen-4-one (Ic)

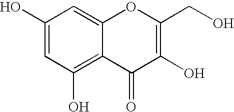

5,7-Dihydroxy-3-methoxy-2-methoxymethylchromone (3 g, 11.9 mmol) is dissolved in methylene chloride (50 ml) at RT with stirring and under an inert gas. The suspension is subsequently cooled to 0° C. before the dropwise addition of BBr₃ (3.39 ml, 35.7 mmol). The yellow suspension is stirred at RT for a further 1 h. The yellow product is filtered off and washed with 32% HCl-acidic water (pH 3-4). The yellow solid is dried at 40° C. in vacuo and subsequently purified by silica gel chromatography with the aid of a solution of methylene chloride and MeOH (methanol) (95/5).

Yield: 1.8 g (48%)

EI-MS: m/e: 224 M⁺

¹H NMR (250 MHz, DMSO): δ (ppm)=4.48 (d, 2H, J=2.75 Hz, CH₂); 5.47 (t, 1H, J=5.5 Hz, OH on CH₂OH); 6.17 (d, 1H, J=1.0 Hz, H-8); 6.33 (d, 1H, J=1.0 Hz, H-6); 9.09 (s, 1H, OH-3); 10.77 (bs, 1H, OH-7); 12.44 (s, 1H, OH-5).

¹³C NMR (75 MHz, DMSO, protons decoupled): δ (ppm)=55.3 (CH₂); 93.3 (CH); 98.1 (CH); 103.6 (Cquat.); 135.9 (Cquat.); 151.6 (Cquat.); 156.6 (Cquat.); 160.9 (Cquat.); 163.9 (Cquat.); 176.3 (Cquat.).

HR-MS: (C₁₀H₈O₆) calc.: 224.0320; found: 224.0323±1.0 ppm.

### Example 4

### Preparation of 5,7-dihydroxy-3-methoxy-2-methylchromen-4-one (Id)

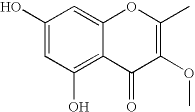

2-Methoxy-1-(2,4,6-trihydroxyphenyl)ethanone (5 g, 25.2 mmol) is dissolved in 150 ml of THF at RT and under an inert gas with stirring. K₂CO₃ (10.46 g, 65.7 mmol) is added, and the yellow suspension is heated at 50° C. for 30 min. Acetyl chloride (3.78 ml, 53 mmol) is added dropwise to the warm suspension, and the mixture is heated under reflux, i.e. at 66° C., for 20 h. The solution is subsequently cooled to RT and poured into ice-water. The mixture is neutralised (pH 7-8) using HCl (32%) and then extracted with ethyl acetate. The organic phase is dried using Na₂SO₄, filtered and evaporated. Recrystallisation is carried out from dichloromethane.

Yield: 2.8 g (50%)

EI-MS: m/e: 222 M⁺

¹H NMR (250 MHz, DMSO): δ (ppm)=2.37 (s, 3H, CH₃-2); 3.75 (s, 3H, OCH₃); 6.17 (d, 1H, J=2.0 Hz, H-8); 6.32 (d, 1H, J=2.0 Hz, H-6); 10.70 (bs, 1H, OH-7); 12.60 (s, 1H, OH-5).

¹³C NMR (75 MHz, DMSO, protons decoupled): δ (ppm)=15.0 (CH₃); 60.0 (CH₃); 93.5 (CH); 98.5 (CH); 104.3 (Cquat.); 156.6 (Cquat.); 160.0 (Cquat.); 161.3 (Cquat.); 163.9 (Cquat.); 177.0 (Cquat.).

HR-MS: (C₁₁H₁₀O₅) calc.: 222.0528; found: 222.0524±1.9 ppm.

### Example 5

### Preparation of 2-methyl-3,5,7-trihydroxychromen-4-one (Ie)

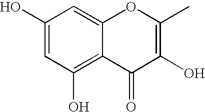

5,7-Dihydroxy-3-methoxy-2-methylchromone (1 g, 4.5 mmol) is suspended in 10 ml of methylene chloride at RT with stirring and under an inert gas. The mixture is cooled to 0° C., before BBr₃ is added dropwise. The mixture is stirred at RT for a further 1 h, and the yellow product is filtered off and washed with an aqueous solution of HCl (0.05 M). The solid is dried at 40° C. in vacuo and recrystallised from dichloromethane (white solid).

Yield: 800 mg (91%)

EI-MS: m/e: 208 M⁺

¹H NMR (250 MHz, DMSO): δ (ppm)=2.33 (s, 3H, CH₃); 6.15 (d, 1H, J=1.0 Hz, H-8); 6.30 (d, 1H, J=1.0 Hz, H-6); 8.84 (bs, 1H, OH-3); 10.68 (bs, 1H, OH-7); 12.54 (s, 1H, OH-5).

¹³C NMR (75 MHz, DMSO, protons decoupled): δ (ppm)=14.7 (CH₃); 93.2 (CH); 98.1 (CH); 103.4 (Cquat.); 136.0 (Cquat.); 151.1 (Cquat.); 156.4 (Cquat.); 160.9 (Cquat.); 163.4 (Cquat.); 175.4 (Cquat.); 177.0 (Cquat.).

HR-MS: (C₁₀H₈O₅) calc.: 208.0371; found: 208.0375±1.8 ppm.

### Example 6

### Preparation of 7-hydroxychromen-4-one (If)

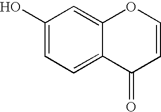

3-Formyl-7-hydroxychromone (300 mg, 1.58 mmol) is suspended in water (80 ml) at 50° C. with stirring. K₂CO₃ (654 mg, 4.73 mmol) is added, and the mixture is boiled under reflux for 30 min. The brown solution is cooled to room temperature or then to 0° C. and 32% HCl is added for neutralisation (pH 6-7). A pale-brown solid is formed, which is filtered off and dried at 40° C. in vacuo. The mother liquor is extracted with ethyl acetate, and the organic phases are dried using Na₂SO₄, filtered and evaporated, giving a dark-brown compound. The product is purified by silica gel chromatography using a mixture of methylene chloride and MeOH (97/3), giving a pale-yellow solid.

Yield: 90 mg (35%)

EI-MS: m/e: 162 M⁺

¹H NMR (250 MHz, DMSO): δ (ppm)=6.20 (d, 1H, J=5.75 Hz, H-3); 6.84 (d, 1H, J=2.0 Hz, H-8); 6.91 (dd, 1H, J=8.5 Hz, J=2.0 Hz, H-6); 7.87 (d, 1H, J=8.5 Hz, H-5); 8.13 (d, 1H, J=5.75 Hz, H-2); 10.73 (bs, 1H, OH).

¹³C NMR (75 MHz, DMSO, protons decoupled): δ (ppm)=102.3 (CH); 111.9 (CH); 115.0 (CH); 117.0 (Cquat.); 126.6 (CH); 156.0 (CH); 157.7 (Cquat.); 162.5 (CH); 175.5 (Cquat.).

HR-MS: (C₉H₆O₃) calc.: 162.0317; found: 162.0316±0.1 ppm.

### Example 7

### Preparation of 7,8-dihydroxychromen-4-one (Ig)

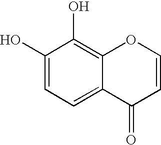

3-Formyl-7,8-dihydroxychromone (500 mg, 2.42 mmol) is suspended in water (100 ml) at 50° C. with stirring. K₂CO₃ (1.0 g, 7.27 mmol) is added, and the suspension is boiled under reflux for 30 min. The brown solution is cooled to RT and then to 0° C., and 32% HCl is added dropwise for neutralisation (pH 6-7). A dark-brown solid is formed, which is filtered off and dried at 40° C. in vacuo. The mother liquor is extracted with ethyl acetate, and the organic phases are dried using Na₂SO₄, filtered and evaporated, giving a dark-brown compound. The product is purified by silica gel chromatography using a mixture of methylene chloride and MeOH (95/5), giving a pale-yellow solid.

Yield: 20 mg (47%)

EI-MS: m/e: 178 M⁺

¹H NMR (250 MHz, DMSO): δ (ppm)=6.17 (d, 1H, J=5.75 Hz, H-3); 6.93 (d, 1H, J=8.5 Hz, H-6); 7.37 (d, 1H, J=8.5 Hz, H-5); 8.17 (d, 1H, J=5.75 Hz, H-2); 9.36 (bs, 1H, OH-8); 10.25 (bs, 1H, OH-7).

¹³C NMR (75 MHz, DMSO, protons decoupled): δ (ppm)=111.3 (CH); 113.9 (CH); 115.0 (CH); 117.8 (Cquat.); 133.0 (Cquat.); 146.9 (Cquat.); 150.1 (Cquat.); 155.8 (CH); 176.0 (Cquat.).

HR-MS: (C₉H₆O₄) calc.: 178.0266; found: 178.0264±0.8 ppm.

### Example 8

### Melanogenesis Assay

The action of the compounds of the formula I as tanning agents is tested through the ability to increase melanin synthesis. The reference used is kojic acid, which acts on melanin synthesis and prevents a possible brown coloration of the skin. It can be seen from Table 1 that the compounds Ia to 1 g according to the invention are distinguished by the fact that—after topical application—they induce the formation of skin-inherent pigments in the skin, increase melanin synthesis in the skin and in this way produce enhanced tanning of the skin. They have no adverse health effects, are non-irritant and easy to handle, and the resultant hue naturally corresponds to that of the natural healthy skin colour. The tanning obtained is light-fast and cannot be washed off since it corresponds to natural tanning.

To this end, primary normal human melanocytes (R6-NHEM-2)* are cultivated in 24 microtitre plates (96-well, 120,000 cells per tube). Two additional plates are carried out, one for evaluation of the viability of the cells (standard MTT assay) and a second for protein investigation (using a calorimetric standard protocol). After 50% confluence, the culture medium is replaced by fresh culture medium which comprises either the control solution (KA or IBMX) or the 7 solutions to be investigated. Each experiment is repeated 3 times. The cells are incubated at 37° C. (and 5% CO₂) for 240 hours, during which the medium is changed every 3 days. After incubation, monolayers are rinsed, the cells are destroyed, and melanin is extracted by means of a 0.5 M NaOH solution. *NHEM-2=normal human epidermal melanocytes (cell type)

The optical density (OD) of each experimentally determined value is measured at 405 nm against the melanin standard (standard curve 0.39 to 100 μg/ml of melanin, Sigma M8631). The measurement results are quoted in μg/ml of melanin and in percent of the negative untreated control.

TABLE 1
Results of the melanogenesis assay

| | | Melanin | |
|---|---|---|---|
| Compounds | Concentration | μg/ml | % of control |
| IBMX¹⁾ | 200 μM | 15.39 | 166 |
| Kojic acid | 560 μM | 5.55 | 55 |
| 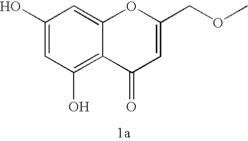 | 450 μM 150 μM 50 μM | 19.98 22.14 17.87 | 215 239 192 |
| 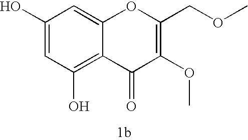 | 200 μM 40 μM 8 μM | 25.2 23.7 20.9 | 128 120 106 |
| 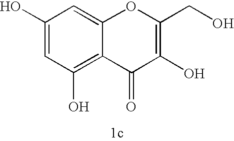 | 200 μM 40 μM 8 μM | 27.6 23.8 20.8 | 139 120 105 |
| 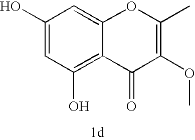 | 40 μM 8 μM 1.6 μM | 27.0 22.0 20.8 | 137 112 106 |
| 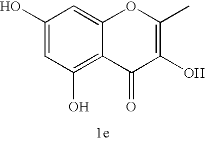 | 200 μM 40 μM 8 μM | 27.2 23.9 20.6 | 138 121 104 |
| 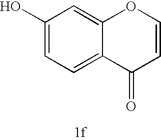 | 40 μM 8 μM 1.6 μM | 24.7 20.3 19.9 | 124 102 100 |
| 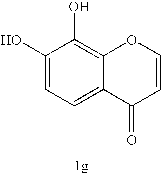 | 8 μM 1.6 μM 0.32 μM | 19.8 19.9 20.7 | 100 100 104 |

¹⁾IBMX denotes 3-isobutyl-1-methylxanthine

### Example 9

### Compositions

Recipes for cosmetic compositions which comprise compounds according to Examples 1-3 are indicated by way of example below. In addition, the INCI names of the commercially available compounds are indicated.

UV-Pearl, OMC stands for the composition with the INCI name:

Water (for EU: Aqua), Ethylhexyl Methoxycinnamate, Silica, PVP, Chlorphenesin, BHT; this composition is commercially available under the name Eusolex®UV PearITMOMC from Merck KGaA, Darmstadt.

The other UV-Pearl products indicated in the tables are in each case of analogous composition, with OMC being replaced by the UV filters indicated.

TABLE 1
W/O emulsions (data in % by weight)

| | 1-1 | 1-2 | 1-3 | 1-4 | 1-5 | 1-6 | 1-7 | 1-8 | 1-9 | 1-10 |
|---|---|---|---|---|---|---|---|---|---|---|
| Titanium Dioxide | | 2 | 5 | | | | | | | 3 |
| 5,7-Dihydroxy-3-methoxy-2- | 5 | 3 | 2 | 1 | 2 | | | | 1 | 1 |
| methylchromen-4-one | | | | | | | | | | |
| 5,7-Dihydroxy-3-methoxy-2- | | | | | | 1 | 2 | 1 | | |
| methoxymethylchromone | | | | | | | | | | |
| Zinc Oxide | | | | | | | | 5 | 2 | |
| UV-Pearl, OMC | 30 | 15 | 15 | 15 | 15 | 15 | 15 | 15 | 15 | 15 |
| Polyglyceryl 3-Dimerate | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 |
| Caprylic/Capric Triglyceride | 7 | 7 | 7 | 7 | 7 | 7 | 7 | 7 | 7 | 7 |
| Hexyl Laurate | 4 | 4 | 4 | 4 | 4 | 4 | 4 | 4 | 4 | 4 |
| PVP/Eicosene Copolymer | 2 | 2 | 2 | 2 | 2 | 2 | 2 | 2 | 2 | 2 |
| Propylene Glycol | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 | 0.5 |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Water | to | to | to | to | to | to | to | to | to | to |
| | 100 | 100 | 100 | 100 | 100 | 100 | 100 | 100 | 100 | 100 |

| | 1-11 | 1-12 | 1-13 | 1-14 | 1-15 | 1-16 | 1-17 | 1-18 |
|---|---|---|---|---|---|---|---|---|
| Titanium Dioxide | 3 | | 2 | | 3 | | 2 | 5 |
| Benzylidene Malonate Polysiloxane | | 1 | 0.5 | | | | | |
| Methylene Bis-benzotriazolyl | 1 | 1 | 0.5 | | | | | |
| Tetramethylbutylphenol | | | | | | | | |
| 5,7-Dihydroxy-3-methoxy-2-methoxy- | 5 | 3 | 2 | 5 | 1 | 3 | 7 | 2 |
| methylchromone | | | | | | | | |
| Polyglyceryl 3-Dimerate | 3 | 3 | 3 | 3 | | | | |
| Cera Alba | 0.3 | 0.3 | 0.3 | 0.3 | 2 | 2 | 2 | 2 |
| Hydrogenated Castor Oil | 0.2 | 0.2 | 0.2 | 0.2 | | | | |
| Paraffinium Liquidum | 7 | 7 | 7 | 7 | | | | |
| Caprylic/Capric Triglyceride | 7 | 7 | 7 | 7 | | | | |
| Hexyl Laurate | 4 | 4 | 4 | 4 | | | | |
| PVP/Eicosene Copolymer | 2 | 2 | 2 | 2 | | | | |
| Propylene Glycol | 4 | 4 | 4 | 4 | | | | |
| Magnesium Sulfate | 0.6 | 0.6 | 0.6 | 0.6 | | | | |
| Tocopherol | 0.5 | 0.5 | 0.5 | 0.5 | | | | |
| Tocopheryl Acetate | 0.5 | 0.5 | 0.5 | 0.5 | 1 | 1 | 1 | 1 |
| Cyclomethicone | 0.5 | 0.5 | 0.5 | 0.5 | | | | |
| Propylparaben | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 |
| Dicocoyl Pentaerythrityl Citrate (and) | | | | | 6 | 6 | 6 | 6 |
| Sorbitan Sesquioleate (and) Cera | | | | | | | | |
| Alba (and) Aluminium Stearate | | | | | | | | |
| PEG-7 Hydrogenated Castor Oil | | | | | 1 | 1 | 1 | 1 |
| Zinc Stearate | | | | | 2 | 2 | 2 | 2 |
| Oleyl Erucate | | | | | 6 | 6 | 6 | 6 |
| Decyl Oleate | | | | | 6 | 6 | 6 | 6 |
| Dimethicone | | | | | 5 | 5 | 5 | 5 |
| Tromethamine | | | | | 1 | 1 | 1 | 1 |
| Glycerine | | | | | 5 | 5 | 5 | 5 |
| Allantoin | | | | | 0.2 | 0.2 | 0.2 | 0.2 |
| Water | to | to | to | to | to | to | to | to |
| | 100 | 100 | 100 | 100 | 100 | 100 | 100 | 100 |

| | 1-19 | 1-20 | 1-21 | 1-22 | 1-23 | 1-24 | 1-25 | 1-26 | 1-27 | 1-28 | 1-29 |
|---|---|---|---|---|---|---|---|---|---|---|---|
| Titanium Dioxide | | 2 | 5 | | | | | | | 3 | 3 |
| Benzylidene Malonate Polysiloxane | | | | 1 | | | | | 1 | 1 | |
| Zinc Oxide | | | | | | | | 5 | 2 | | |
| 5,7-Dihydroxy-3-methoxy-2-methyl- | 5 | 5 | 5 | 5 | 7 | 5 | 5 | 5 | 5 | 5 | 8 |
| chromen-4-one | | | | | | | | | | | |
| UV-Pearl, OCR | | 10 | | | | | | | | | 5 |
| UV-Pearl, EthylhexylDimethylPABA | | | 10 | | | | | | | | |
| UV-Pearl, Homosalate | | | | 10 | | | | | | | |
| UV-Pearl, Ethylhexyl Salicylate | | | | | 10 | | | | | | |
| UV-Pearl, OMC, BP-3 | | | | | | 10 | | | | | |
| UV-Pearl, OCR, BP-3 | | | | | | | 10 | | | | |
| UV-Pearl, Ethylhexyl Dimethyl | | | | | | | | 10 | | | |
| PABA, BP-3 | | | | | | | | | | | |
| UV-Pearl, Homosalate, BP-3 | | | | | | | | | 10 | | |
| UV-Pearl, Ethylhexyl Salicylate, | | | | | | | | | | 10 | |
| BP-3 | | | | | | | | | | | |
| BMDBM | | | | | | | | | | | 2 |
| UV-Pearl, OMC, | 25 | | | | | | | | | | |
| 4-Methylbenzylidene Camphor | | | | | | | | | | | |
| Polyglyceryl 3-Dimerate | 3 | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 | 7 |
| Caprylic/Capric Triglyceride | 7 | 7 | 7 | 7 | 7 | 7 | 7 | 7 | 7 | 7 | 7 |
| Hexyl Laurate | 4 | 4 | 4 | 4 | 4 | 4 | 4 | 4 | 4 | 4 | 4 |
| PVP/Eicosene Copolymer | 2 | 2 | 2 | 2 | 2 | 2 | 2 | 2 | 2 | 2 | 2 |
| Propylene Glycol | 4 | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 | 0.5 | 0.5 |
| Propylparaben | 0.05 | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 | 0.15 |

| | |
|---|---|
| Water | to 100 |

TABLE 2
O/W emulsions, data in % by weight

| | 2-1 | 2-2 | 2-3 | 2-4 | 2-5 | 2-6 | 2-7 | 2-8 | 2-9 | 2-10 |
|---|---|---|---|---|---|---|---|---|---|---|
| Titanium Dioxide | | 2 | 5 | | | | | | | 3 |
| Methylene Bis-benzotriazolyl | | | | | | 1 | 2 | 1 | | |
| Tetramethylbutylphenol | | | | | | | | | | |
| 5,7-Dihydroxy-3-methoxy-2- | | | | 1 | 2 | | | | 1 | 1 |
| methoxymethylchromone | | | | | | | | | | |
| 4′-Methoxy-6-hydroxyflavone | 1 | 3 | | 2 | | 5 | | 5 | 2 | |
| 5,7-Dihydroxy-3-methoxy-2-methyl- | 5 | 5 | 5 | 5 | 5 | 5 | 5 | 5 | 5 | 5 |
| chromen-4-one | | | | | | | | | | |
| 4-Methylbenzylidene Camphor | 2 | | 3 | | 4 | | 3 | | 2 | |
| BMDBM | 1 | 3 | | 3 | 3 | | 3 | 3 | 3 | |
| Stearyl Alcohol (and) Steareth-7 | 3 | 3 | 3 | 3 | 3 | 3 | 3 | 3 | 3 | 3 |
| (and) Steareth-10 | | | | | | | | | | |
| Glyceryl Stearate (and) Ceteth- | 3 | 3 | 3 | 3 | 3 | 3 | 3 | 3 | 3 | 3 |
| 20 | | | | | | | | | | |
| Glyceryl Stearate | 3 | 3 | 3 | 3 | 3 | 3 | 3 | 3 | 3 | 3 |
| Microwax | 1 | 1 | 1 | 1 | 1 | 1 | 1 | 1 | 1 | 1 |
| Cetearyl Octanoate | 11.5 | 11.5 | 11.5 | 11.5 | 11.5 | 11.5 | 11.5 | 11.5 | 11.5 | 11.5 |
| Caprylic/Capric Triglyceride | 6 | 6 | 6 | 6 | 6 | 6 | 6 | 6 | 6 | 6 |
| Oleyl Oleate | 6 | 6 | 6 | 6 | 6 | 6 | 6 | 6 | 6 | 6 |
| Propylene Glycol | 4 | 4 | 4 | 4 | 4 | 4 | 4 | 4 | 4 | 4 |
| Glyceryl Stearate SE | | | | | | | | | | |
| Stearic Acid | | | | | | | | | | |
| Persea Gratissima | | | | | | | | | | |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Tromethamine | | | 1.8 | | | | | | | |
| Glycerine | | | | | | | | | | |
| Water | to | to | to | to | to | to | to | to | to | to |
| | 100 | 100 | 100 | 100 | 100 | 100 | 100 | 100 | 100 | 100 |

| | 2-11 | 2-12 | 2-13 | 2-14 | 2-15 | 2-16 | 2-17 | 2-18 |
|---|---|---|---|---|---|---|---|---|
| Titanium Dioxide | 3 | | 2 | | | | 2 | 5 |
| Benzylidene Malonate Polysiloxane | | 1 | 0.5 | | | | | |
| Methylene Bis-benzotriazolyl | 1 | 1 | 0.5 | | | | | |
| Tetramethylbutylphenol | | | | | | | | |
| 4′-Methoxy-7-β-glucoside Flavone | | | | 1 | 2 | | | |
| 5,7-Dihydroxy-3-methoxy-2-methoxy- | 1 | 3 | | 2 | | 5 | | 5 |
| methylchromone | | | | | | | | |
| 5,7-Dihydroxy-3-methoxy-2-methyl- | 5 | 5 | 5 | 5 | 5 | 5 | 5 | 5 |
| chromen-4-one | | | | | | | | |
| 5,7-Dihydroxy-3-hydroxy-2-hydroxy- | 1 | 5 | 4 | | 6 | | 7 | |
| methylchromone | | | | | | | | |
| Zinc Oxide | | | 2 | | | | | |
| UV-Pearl, OMC | 15 | 15 | 15 | 30 | 30 | 30 | 15 | 15 |
| 4-Methylbenzylidene Camphor | | | | 3 | | | | |
| BMDBM | | | | 1 | | | | |
| Phenylbenzimidazole Sulfonic Acid | | | | | 4 | | | |
| Stearyl Alcohol (and) Steareth-7 | 3 | 3 | 3 | 3 | | | | |
| (and) Steareth-10 | | | | | | | | |
| Glyceryl Stearate (and) Ceteth-20 | 3 | 3 | 3 | 3 | | | | |
| Glyceryl Stearate | 3 | 3 | 3 | 3 | | | | |
| Microwax | 1 | 1 | 1 | 1 | | | | |
| Cetearyl Octanoate | 11.5 | 11.5 | 11.5 | 11.5 | | | | |
| Caprylic/Capric Triglyceride | 6 | 6 | 6 | 6 | 14 | 14 | 14 | 14 |
| Oleyl Oleate | 6 | 6 | 6 | 6 | | | | |
| Propylene Glycol | 4 | 4 | 4 | 4 | | | | |
| Glyceryl Stearate SE | | | | | 6 | 6 | 6 | 6 |
| Stearic Acid | | | | | 2 | 2 | 2 | 2 |
| Persea Gratissima | | | | | 8 | 8 | 8 | 8 |
| Propylparaben | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 |
| Tromethamine | | | | | 1.8 | | | |
| Glycerine | | | | | 3 | 3 | 3 | 3 |
| Water | to | to | to | to | to | to | to | to |
| | 100 | 100 | 100 | 100 | 100 | 100 | 100 | 100 |

| | 2-19 | 2-20 | 2-21 | 2-22 | 2-23 | 2-24 | 2-25 | 2-26 | 2-27 | 2-28 |
|---|---|---|---|---|---|---|---|---|---|---|
| Titanium Dioxide | | | | | | | 3 | 3 | | 2 |
| Benzylidene Malonate Poly- | 1 | 2 | | | | 1 | 1 | | 1 | 0.5 |
| siloxane | | | | | | | | | | |
| 7,8,3′,4′-Tetrahydroxyflavone | | | | 1 | 2 | | | | 1 | 1 |
| 5,7-Dihydroxy-3-methoxy-2- | 1 | 3 | | 2 | | 5 | | 5 | 2 | |
| methylchromen-4-one | | | | | | | | | | |
| 7,8-Dihydroxychromen-4-one | 5 | 5 | 5 | 5 | 5 | 5 | 5 | 5 | 5 | 5 |
| Methylene Bis-benzotriazolyl | | | 1 | 2 | 1 | | | 1 | 1 | 0.5 |
| Tetramethylbutylphenol | | | | | | | | | | |
| Zinc Oxide | | | | | 5 | 2 | | | | 2 |
| UV-Pearl, OMC | 15 | 15 | 15 | 15 | 15 | 15 | 15 | 15 | 15 | 15 |
| Caprylic/Capric Triglyceride | 14 | 14 | 14 | 14 | 14 | 14 | 14 | 14 | 14 | 14 |
| Oleyl Oleate | | | | | | | | | | |
| Propylene Glycol | | | | | | | | | | |
| Glyceryl Stearate SE | 6 | 6 | 6 | 6 | 6 | 6 | 6 | 6 | 6 | 6 |
| Stearic Acid | 2 | 2 | 2 | 2 | 2 | 2 | 2 | 2 | 2 | 2 |
| Persea Gratissima | 8 | 8 | 8 | 8 | 8 | 8 | 8 | 8 | 8 | 8 |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Glyceryl Stearate, Ceteareth- | | | | | | | | | | |
| 20, Ceteareth-10, Cetearyl | | | | | | | | | | |
| Alcohol, Cetyl Palmitate | | | | | | | | | | |
| Ceteareth-30 | | | | | | | | | | |
| Dicaprylyl Ether | | | | | | | | | | |
| Hexyldecanol, Hexyldexyl | | | | | | | | | | |
| Laurate | | | | | | | | | | |
| Cocoglycerides | | | | | | | | | | |
| Tromethamine | | | | | | | | | | |
| Glycerine | 3 | 3 | 3 | 3 | 3 | 3 | 3 | 3 | 3 | 3 |
| Water | to | to | to | to | to | to | to | to | to | to |
| | 100 | 100 | 100 | 100 | 100 | 100 | 100 | 100 | 100 | 100 |

TABLE 3
Gels, data in % by weight

| | 3-1 | 3-2 | 3-3 | 3-4 | 3-5 | 3-6 | 3-7 | 3-8 | 3-9 | 3-10 |
|---|---|---|---|---|---|---|---|---|---|---|
| a = aqueous gel | | | | | | | | | | |
| Titanium Dioxide | | 2 | 5 | | | | | | | 3 |
| 7,8-Dihydroxychromen-4-one | | | | 1 | 2 | | | | 1 | 1 |
| 2-Methyl-3,5,7-trihydroxychromen-4- | 1 | 3 | | 2 | | 5 | | 5 | 2 | |
| one | | | | | | | | | | |
| Benzylidene Malonate Polysiloxane | | | 1 | 1 | 2 | | | | 1 | 1 |
| Methylene Bis-benzotriazolyl | | 1 | | | | 1 | 2 | 1 | | |
| Tetramethylbutylphenol | | | | | | | | | | |
| Zinc Oxide | | | | 2 | | | | 5 | 2 | |
| UV-Pearl, Ethylhexyl Methoxy- | 30 | 15 | 15 | 15 | 15 | 15 | 15 | 15 | 15 | 15 |
| cinnamate | | | | | | | | | | |
| 4-Methylbenzylidene Camphor | | | | | 2 | | | | | |
| Butylmethoxydibenzoylmethane | | 1 | | | | | | | | |
| Phenylbenzimidazole Sulfonic Acid | | | 4 | | | | | | | |
| Prunus Dulcis | 5 | 5 | 5 | 5 | 5 | 5 | 5 | 5 | 5 | 5 |
| Tocopheryl Acetate | 0.5 | 0.5 | 0.5 | 0.5 | 0.5 | 0.5 | 0.5 | 0.5 | 0.5 | 0.5 |
| Caprylic/Capric Triglyceride | 3 | 3 | 3 | 3 | 3 | 3 | 3 | 3 | 3 | 3 |
| Octyldodecanol | 2 | 2 | 2 | 2 | 2 | 2 | 2 | 2 | 2 | 2 |
| Decyl Oleate | 2 | 2 | 2 | 2 | 2 | 2 | 2 | 2 | 2 | 2 |
| PEG-8 (and) Tocopherol (and) | 0.05 | 0.05 | 0.05 | 0.05 | 0.05 | 0.05 | 0.05 | 0.05 | 0.05 | 0.05 |
| Ascorbyl Palmitate (and) Ascorbic | | | | | | | | | | |
| Acid (and) Citric Acid | | | | | | | | | | |
| Sorbitol | 4 | 4 | 4 | 4 | 4 | 4 | 4 | 4 | 4 | 4 |
| Polyacrylamide (and) C13-14 | 3 | 3 | 3 | 3 | 3 | 3 | 3 | 3 | 3 | 3 |
| Isoparaffin (and) Laureth-7 | | | | | | | | | | |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Tromethamine | | | 1.8 | | | | | | | |
| Water | to | to | to | to | to | to | to | to | to | to |
| | 100 | 100 | 100 | 100 | 100 | 100 | 100 | 100 | 100 | 100 |

### Example 10

### Self-Tanning Cream Comprising DHA (O/W)

The self-tanning cream is prepared by heating phase A (consisting of glyceryl stearate, stearyl alcohol, cetearyl alcohol, cetearyl ethylhexanoate, caprylic triglyceride, stearoxydimethicone, dimethicone, tocopheryl acetate, propylparaben) and phase B (consisting of propylene glycol, methylparaben and water). Phase B is slowly stirred into phase A, and the mixture is homogenised. The mixture is cooled with stirring. The 5,7-dihydroxy-3-methoxy-2-methylchromen-4-one is dissolved in the water before the DHA. Phase C (consisting of DHA, 5,7-dihydroxy-3-methoxy-2-methylchromen-4-one and water) is added at 40° C.

| INCI | [%] |
|---|---|
| Aqua (Water) | 69.50 |
| Dihydroxyacetone | 2.00 |
| 5,7-Dihydroxy-3-methoxy-2- | 0.50 |
| methylchromen-4-one | |
| Methylparaben | 0.15 |
| Propylene glycol | 3.00 |
| Glyceryl stearate, Stearyl | 8.00 |
| alcohol | |
| CETETH-20, STEARETH-25 | |
| Cetearyl alcohol | 1.50 |
| Cetearyl ethylhexanoate | 6.50 |
| Caprylic/Capric triglyceride | 6.50 |
| Stearoxy dimethicone | 1.20 |
| Dimethicone | 0.50 |
| Tocopheryl acetate | 0.50 |
| Propylparaben | 0.05 |
| Perfume | 0.10 |

1. Use of at least one compound of the formula I 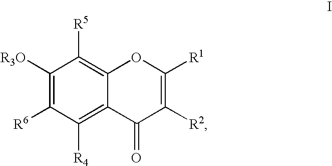
where
R¹ and R² may be identical or different and stand for H, OH, OCH₃, CH₃, CH₂OH or CH₂OCH₃,
R³ stands for H or straight-chain or branched C₁- to C₂₀-alkyl groups,
R⁴ stands for H or OR⁸,
R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain, and furthermore the alkyl chain may also be interrupted by oxygen, and
R⁸ stands for H or straight-chain or branched C₁- to C₂₀-alkyl groups, with the proviso that R does not denote H if R¹ denotes CH₃, as self-tanning substance.

2. Use according to claim 1, characterised in that R¹ in formula I stands for CH₂OCH₃, CH₂OH or CH₃.

3. Use according to claim 1, characterised in that R² stands for H, OH or OCH₃.

4. Use according to claim 1, characterised in that R³ stands for H and R⁴ stands for H or OH.

5. Use according to claim 1, characterised in that R⁵ stands for H or OH and R⁶ stands for H.

6. Use according to claim 1, characterised in that the compound of the formula I is a compound selected from the compounds having the formulae Ia to Ig:

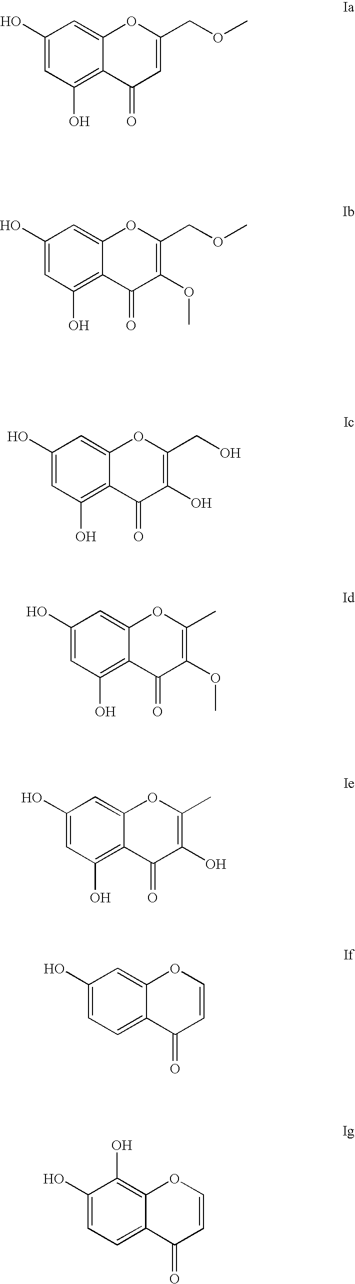

7. Use of the compounds of the formula I according to claim 1 for protecting the skin against harmful UV rays.

8. Use of the compounds of the formula I according to claim 1 for increasing melanin synthesis in the skin.

9. Compounds of the formula I 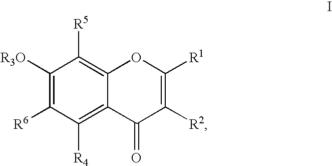
where
R¹ denotes CH₂OH or CH₂OCH₃,
R² denotes OH or OCH₃,
R³ stands for H or straight-chain or branched C₁- to C₂₀-alkyl groups,
R⁴ stands for H or OR⁸,
R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain, and furthermore the alkyl chain may also be interrupted by oxygen, and
R⁸ stands for H or straight-chain or branched C₁- to C₂₀-alkyl groups.

10. Compounds according to claim 9, characterised in that R³ stands for H.

11. Compounds according to claim 9, characterised in that R⁴ stands for H or OH.

12. Compounds according to claim 9, characterised in that R⁵ stands for H or OH and R⁶ stands for H.

13. Compounds according to claim 9, selected from the compounds of the formulae Ib and Ic

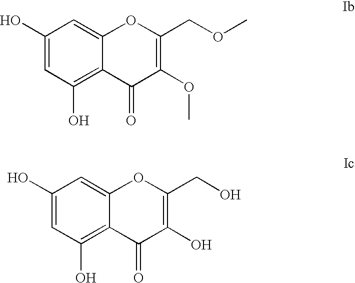

14. Cosmetic and/or dermatological composition comprising at least one compound of the formula I according to claim 9 and at least one vehicle which is suitable for topical applications.

15. Self-tanning composition comprising at least one compound of the formula I according to claim 1 and at least one vehicle which is suitable for topical applications.

16. Composition according to claim 1, characterised in that it comprises one or more compounds of the formula I in an amount of 0.01 to 10% by weight.

17. Composition according to claim 14, characterised in that at least one further self-tanning substance is present.

18. Composition according to claim 17, characterised in that the self-tanning substance is selected from the group glycerolaldehyde, hydroxymethylglyoxal, γ-dialdehyde, erythrulose, 6-aldo-D-fructose, ninhydrin, 5-hydroxy-1,4-naphthoquinone, 2-hydroxy-1,4-naphthoquinone, 1,3-diyhydroxyacetone, dihydroxyacetone phosphate, glyceraldehyde phosphate and erythrose.

19. Composition according to claim 14, characterised in that one or more antioxidants are present.

20. Composition according to claim 14, characterised in that one or more vitamins are present.

21. Composition according to claim 14, characterised in that one or more UV filters are present.

22. Process for the preparation of a composition according to claim 14, characterised in that at least one compound of the formula I is mixed with a vehicle which is suitable for topical applications.

23. Process for the preparation of a compound of the formula I according to claim 9, characterised in that the preparation is carried out by cyclisation of an o-hydroxyacetophenone with a suitable anhydride or with a suitable acyl chloride.
